(19) United States
(12) Patent Application Publication
(10) Pub. No.: US 2015/0230764 A1
(43) Pub. Date: Aug. 20, 2015

(54) DOSE-REDUCING X-RAY AIMING DEVICE

(71) Applicant: Dental Imaging Technologies Corporation, Hatfield, PA (US)

(72) Inventors: Jeremy Charnegie, Emmaus, PA (US); Timothy J. McDermott, Glenside, PA (US); Lisa A. Ratnow, Downingtown, PA (US); Joseph J. Bria, III, Furlong, PA (US); Carl A. Yacono, Sellersville, PA (US); Adam T. Palermo, Philadelphia, PA (US)

(21) Appl. No.: 14/184,226

(22) Filed: Feb. 19, 2014

(51) Int. Cl. A61B 6/14; A61B 6/08; A61B 6/10; A61B 6/00

(52) U.S. Cl. CPC A61B 6/145; A61B 6/4435; A61B 6/08; A61B 6/107

(57) ABSTRACT

A dose-reducing x-ray aiming device includes an aiming ring having shield with an aperture, at least one arm, and at least one sensor holder. The dose-reducing x-ray aiming ring is generally used to aim a dental x-ray machine and at least partially shield some of the x-rays emitted from the x-ray machine while capturing radiographs. X-rays generally pass through the aperture and then interact with a sensor for capturing the radiographs after passing through the tissue of a patient. In one embodiment, the x-ray aiming device provides an all-in-one aiming ring, which allows a user to capture both periapical and bitewing radiographs with a single device. The aiming device also includes an annular ridge or lip which helps align the x-ray machine with the aiming device.

### BACKGROUND

The present invention relates to dental x-ray imaging, and more specifically to aiming rings used with dental x-ray machines.

Aiming rings are often used during intra-oral x-ray imaging procedures to help align an x-ray source positioned outside of a patient's mouth and an x-ray receptor, for example, a film plate or a filmless, intra-oral sensor.

### SUMMARY

In addition to being used to help ensure proper alignment between an x-ray source and a receptor, some currently available aiming rings have features to limit radiation exposure to the particular area or anatomical feature of interest. These x-ray limiting features include collimators, plates, or fixed shutters that block the passage of x-ray radiation. Often, the aiming ring is connected to a sensor holder which is placed in the patient's mouth and held in place via the clamping force of a patient's bite on a biteblock portion of the sensor holder. In some cases, aiming rings are made using steel plates about 3-4 mm thick. As a consequence of using steel plates of such thickness, the aiming rings are relatively heavy. When used in an imaging procedure these types of aiming rings are uncomfortable to the patient because of the biting force required to maintain the aiming ring in position. Often, the patient must manually support the aiming ring with his or her hands during the imaging procedure in addition to or in place of supporting the aiming ring via a biteblock.

In one aspect, the invention provides an x-ray aiming device that is lighter in weight and which may be held in place simply as a consequence of the force applied by the patient's teeth on a biteblock connected to the aiming device. This increases patient comfort as there is less weight to support via the patient's mouth, does not require the patient to maintain their hand is a fixed position (in addition to biting a biteblock), reduces the possibility that the patient's hand will be exposed to x-ray radiation, and helps limit the area of irradiation to that which is necessary to capture the image of interest.

In one embodiment, the x-ray aiming device includes an aiming ring that has an x-ray shield. The x-ray shield is thin enough to be relatively lightweight, yet thick enough to block most of the x-rays. An encasement covers the x-ray shield. The x-ray shield of the x-ray aiming device also includes an aperture that allows x-rays to pass through the x-ray aiming ring.

In another aspect, the invention provides an x-ray aiming device that includes an aiming ring having an x-ray shield and an encasement covering the x-ray shield. The x-ray shield also includes an aperture in the aiming ring allowing x-rays to pass through the x-ray aiming ring. In one embodiment, first and second mounting tabs are formed as part of the encasement. The aiming device includes a sensor holder configured to hold an x-ray sensor. The sensor holder may have multiple configurations such as a bite wing (or first type of) sensor holder or an anterior (or second type of) sensor holder. A first arm is configured to connect to one of the first and second mounting tabs of the plastic overmold and the first type of sensor holder. A second arm is configured to connect to one of the first and second mounting tabs of the plastic overmold and the second type of sensor holder.

Other aspects of the invention will become apparent by consideration of the detailed description and accompanying drawings.

### BRIEF DESCRIPTION OF THE DRAWINGS

FIG. 1 is a perspective view of a dose-reducing x-ray aiming device embodying the present invention.

FIG. 2 is another view of the aiming device of FIG. 1 illustrating an anterior sensor holder disconnected from a sensor holder arm.

FIG. 3 is another view of the aiming device of FIG. 1 illustrating a bitewing sensor holder disconnected from a sensor holder arm.

FIG. 4 is a front view of the aiming device of FIG. 1 illustrated having the anterior sensor holder in solid lines and the bitewing sensor holder in phantom lines.

FIG. 5 is a cross-sectional view of the aiming ring.

FIG. 6 is a front view of the x-ray shield of the aiming ring.

FIG. 7 is a front view of the plastic overmold that encases the x-ray shield.

FIG. 8 is a sectional view of the overmold of FIG. 7 illustrating one of the mounting tabs.

### DETAILED DESCRIPTION

Before any embodiments of the invention are explained in detail, it is to be understood that the invention is not limited in its application to the details of construction and the arrangement of components set forth in the following description or illustrated in the following drawings. The invention is capable of other embodiments and of being practiced or of being carried out in various ways.

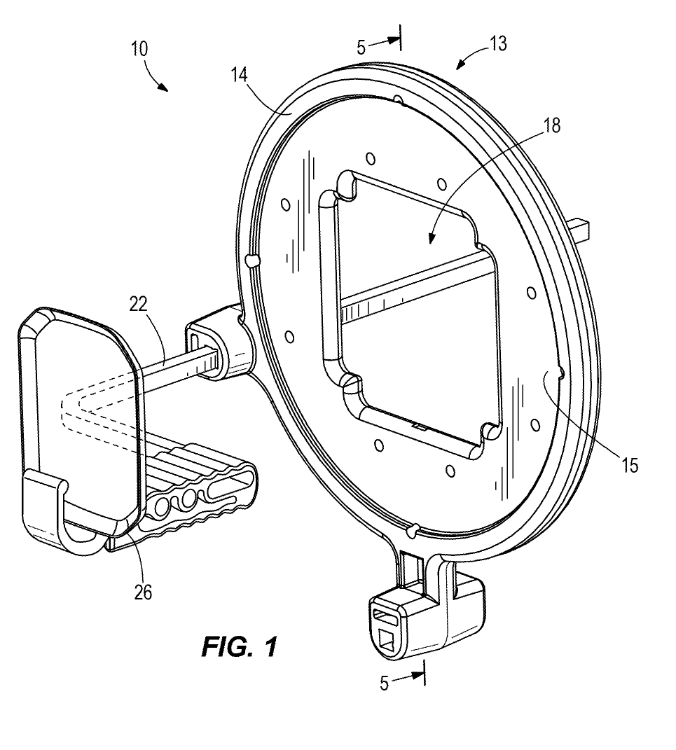

FIG. 1 illustrates a dose-reducing x-ray aiming device 10 according to an embodiment of the invention. The aiming device 10 includes an aiming ring 13 having a raised lip 14 and shield or collimator portion 15. The collimator portion 15 includes an aperture 18. As will be described below, the collimator portion 15 and an annular ridge or raised lip 14 are formed as a result of overmolding a metal shield that has an opening with a plastic material. The aiming device 10 also includes a first arm 22, and a first sensor holder 26 that is designed for capturing a first type of radiograph or x-ray image, for example a periapical image. Alternatively or in addition, the aiming device 10 may also include a second arm 22a and a second sensor holder 26a that is designed for capturing a second type of radiograph or x-ray image, for example, a bitewing image (FIG. 3). Other types of sensor holders or holders designed for more specific types of imaging are possible. For example, instead of or in place of a single periapical sensor holder, an anterior periapical sensor holder and a posterior periapical sensor hold may be used.

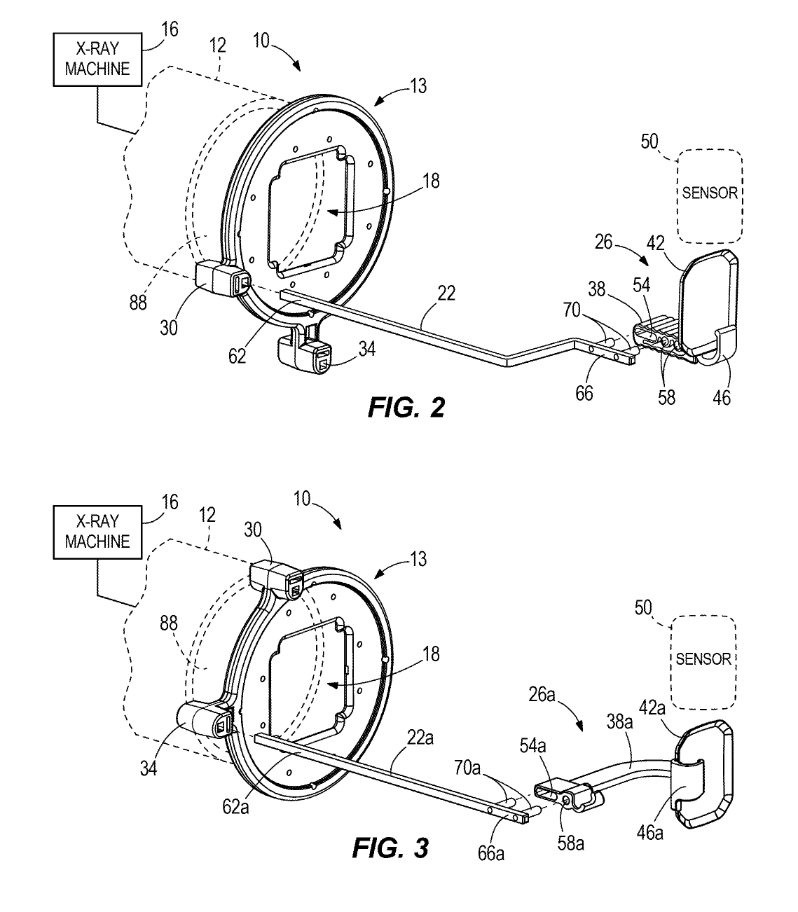

The dose-reducing x-ray aiming device 10 is generally used to aim a dental x-ray source 12 of an x-ray machine 16 (FIGS. 2 and 3) at an area or anatomical feature of interest and at least partially shield or block x-ray radiation emitted from the x-ray source 12 from reaching other areas. In an imaging procedure, x-ray radiation (x-rays) is emitted from the x-ray source 12. X-ray radiation that impinges the collimator portion 15 is prevented from further travel. X-ray radiation passes through the aperture 18, through the object, person, or portion thereof being imaged and impinges a receptor or sensor, which captures x-ray image information. When configured with the applicable arms and sensor holders, the x-ray aiming device 10 provides an all-in-one aiming ring 13, which allows a user to capture both periapical and bitewing radiographs with a single device.

FIG. 2 illustrates the x-ray aiming device 10 with the first arm 22 and the first sensor holder 26. As noted, the first arm 22 and the first sensor holder 26 are used to assist in capturing anterior periapical radiographs or images. The second arm 22a and the second sensor holder 26a illustrated in FIG. 3 are used to assist in capturing bitewing radiographs images. As also noted, the x-ray aiming device 10 may also use a third sensor holder (now shown), which is used to assist in capturing posterior periapical radiographs. The three sensor holders may be color-coded (e.g., blue, yellow, red, etc.) to assist the x-ray operator in choosing the correct sensor holder.

In reference to FIGS. 2 and 3, the aiming ring 13 includes a first mounting tab 30 and a second mount tab 34. The mounting tabs 30, 34 are more specifically anterior/posterior (A/P) and bitewing (BW) mounting tabs and labeled with the indicia A/P and BW in the drawings. The first sensor holder 26 includes a biteblock 38, a face 42, and a sensor clip 46. The sensor clip 46 generally holds a receptor or sensor 50 (for example, an electronic sensor) against the face 42, such that the sensor 50 remains in place during an imaging procedure. The biteblock 38 includes an elongated hole 54 and two round holes 58. The first arm 22 includes a first end 62 and a second end 66. The first end 62 of the first arm 22 engages with the A/P mounting tab 30, and the second end 66 includes two prongs 70 that are received in the holes 54, 58. In normal operation, the x-ray operator places one of the prongs 70 into the round hole 58 nearest the face 42, and the other one of the prongs 70 into the elongated hole 54. Alternatively, if the sensor needs to be positioned further back in the mouth of a patient, the x-ray operator places one of the prongs 70 into the round hole 58 furthest from the face 42.

The second sensor holder 26a includes a biteblock 38a, a face 42a, and a sensor clip 46a. The sensor clip 46a generally holds a sensor 50 against the face 42a, such that the sensor 50 remains in place during an imaging procedure, and the biteblock 38a includes an elongated hole 54a and a round hole 58a. The second arm 22a includes a first end 62a and a second end 66a. The first end 62a engages with the bitewing mounting tab 34, and the second end 66a includes two prongs 70a that are received in the holes 54a, 58a. In normal operation, the x-ray operator places one of the prongs 70a into the round hole 58a, and the other one of the prongs 70a into the elongated hole 54a.

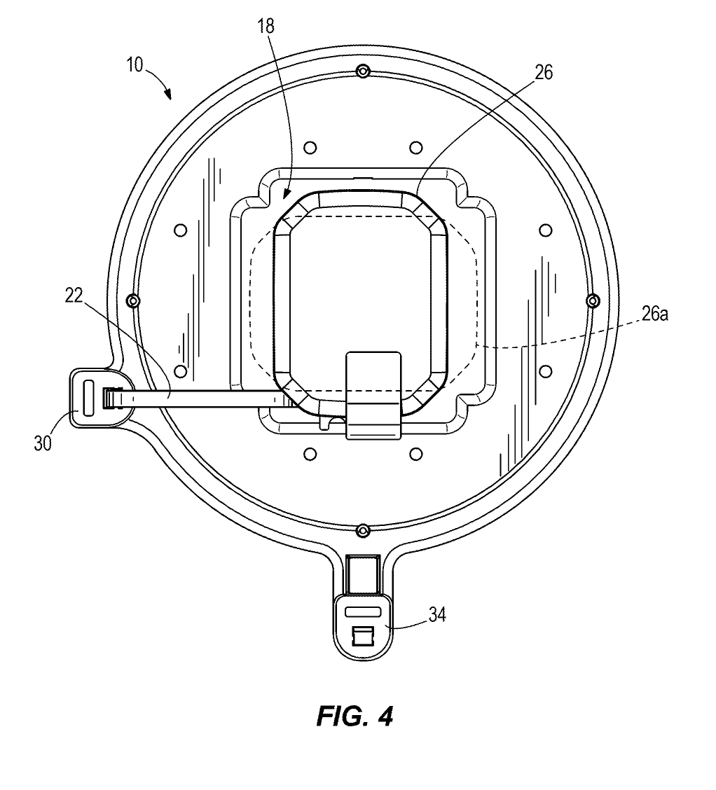

FIG. 4 is a front view of the aiming device and illustrates how the sensor holders 26, 26a align with the aperture 18. The aperture 18 is designed such that it allows x-ray radiation to pass through the aperture 18 to irradiate the sensor 50 illustrated in FIGS. 2 and 3. The aperture 18 is substantially shaped as an outline of two perpendicular elongated rectangles, which correspond to the size of the faces 42, 42a of the sensor holders 26, 26a. This shape helps reduce the dose of x-ray radiation a patient is exposed to during imaging by blocking radiation that is not required to capture the image of interest, but allows the aiming device to be used for two different imaging procedures—bitewing and periapical in which the sensor or receptors are generally aligned in different orientations—landscape and portrait.

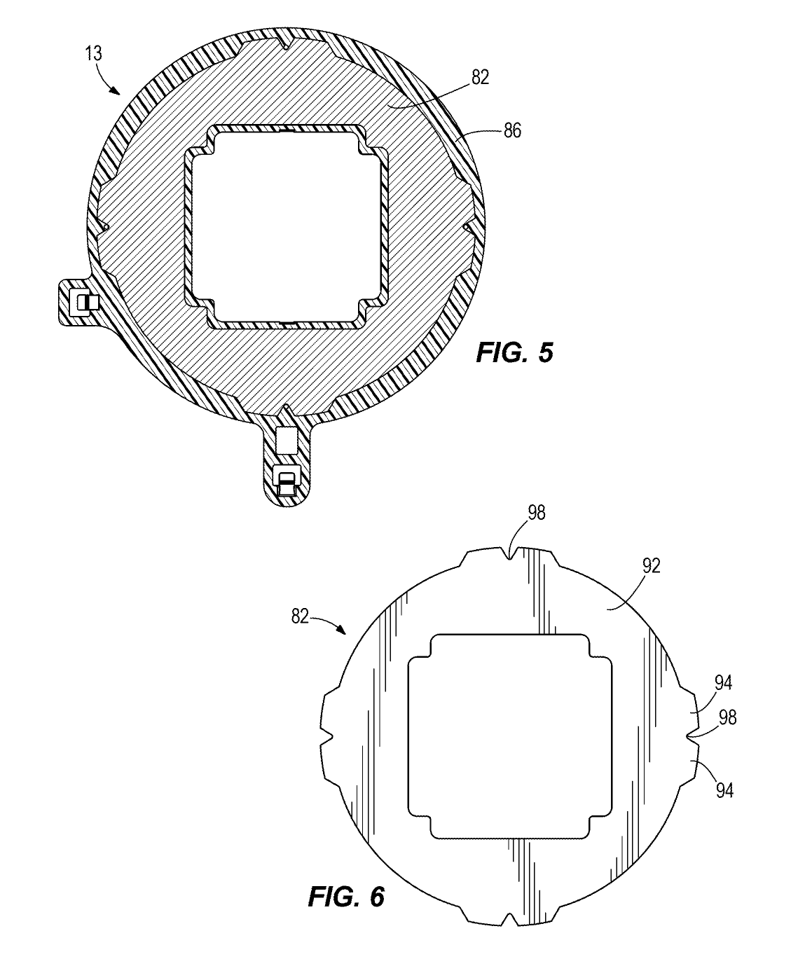

FIGS. 5 and 6 illustrate an x-ray shield 82 and a plastic overmold or encasement 86, which encases the x-ray shield 82. The x-ray shield 82 and the encasement 86 together form the aiming ring 13. In the embodiment illustrated, the x-ray shield 82 is made from a tungsten material. As an example, the tungsten material may be a 97% percent tungsten metal having a thickness of about 0.50 mm. When so constructed, the x-ray shield 82 is lighter than conventional shields made from 3-4 mm-thick steel, yet still capable of blocking x-ray radiation of the type and energy level used in most dental imaging. As a consequence, the aiming ring 13 is relatively light-weight. For example, following the teachings of one embodiment of the invention an aiming ring of approximately 53 grams was produced. In contrast, prior aiming rings with steel plates and exhibiting similar radiation blocking characteristics weighed 88 grams. Thus, in this example, a reduction in weight of 35 grams or approximately 40% was achieved.

The x-ray shield 82 has a flat main surface 92 and is formed with a plurality of tabs 94 located at the outer edge of the x-ray shield 82. The tabs 94 at least partially define a plurality of notches 98 between adjacent tabs 94. The notches 98 of the x-ray shield 82 align with a plurality of stabilizing holes 102 formed in the encasement 86, to be described in detail below.

In the illustrated embodiment, the encasement 86 is an injection molded component that is created using a plastic material. Preferably, the plastic material can withstand repeated x-ray exposure and autoclaving without significant degradation in desired characteristics such as texture, color, shock absorbency, and resistance to or protecting the x-ray shield 82 from environmental elements such as dust, water, and vapor. Additional desirable properties include, for example, high impact strength, resistance to antiseptic cleaning agents, dimensional stability, low water absorption, and biocompatibility. One exemplary, preferred material for the encasement 86 is polyetherimide. Another exemplary, preferred material in polyether ether ketone.

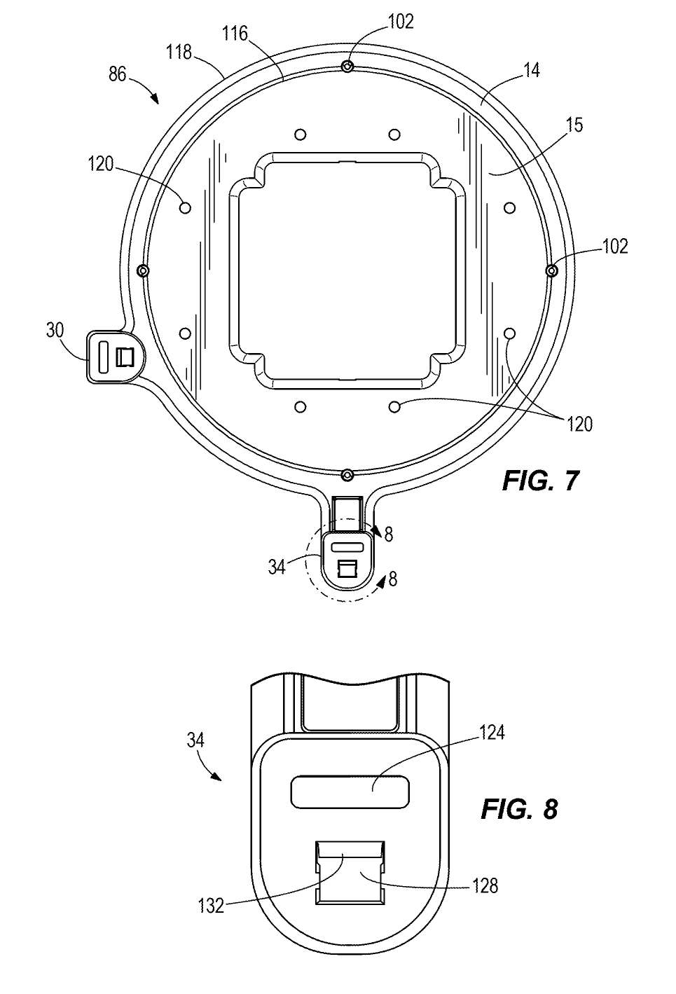

FIG. 7 illustrates the encasement 86 of the aiming ring 13 including the collimator portion 15 and the annular ridge 14. The annular ridge 14 has an inner circumference 116 and an outer circumference 118. The stabilizing holes 102 are formed in alignment with the inner circumference 116 of the annular ridge 14. The encasement 86 also includes a plurality of clamping holes 120 formed in the collimator portion 15. The stabilizing holes 102 and the clamping holes 120 are formed as a result of the molding process. During the molding process, a plurality of stabilizing and clamping pins are positioned to engage the x-ray shield 82 before the plastic is injected into the mold. The stabilizing pins engage with the notches 98 of the x-ray shield 82 to prevent the shield 82 from rotating during the molding process as a result of pressure from the injected plastic. The stabilizing pins are then removed from engagement with the notches 98 leaving the stabilizing holes 102 formed in the plastic overmold. The clamping pins engage the main surface 92 of the x-ray shield 82 to further hold the x-ray shield 82 in place during the molding process. After the plastic is injected into the mold, the clamping pins are moved out of engagement with the main surface 92 of the x-ray shield 82, thereby leaving the clamping holes 120 formed in the encasement 86. Without the use of the pins that form the holes 102, 120, the x-ray shield 82 may become deformed or misaligned due to the pressure of the injection molding process.

Alternatively, or in addition, the encasement 86 can be cast around a supported shield 82. For example, a mixture of resin and hardener, a mixture of two or more different kinds of resins, or a silicone compound (either self-curing and/or with a curing agent) or other elastomer, can be poured into a support mold and left to harden via a chemical reaction, thus forming the encasement 86 without requiring as much heat and pressure as a typical injection molding process.

As an additional method, the encasement 86 can be formed from a two-piece plastic or elastomer assembly with the shield 82 sandwiched between the two plastic or elastomer pieces. The two pieces are then bonded together at least around their edges by, e.g., thermal bonding, ultrasonic welding, or an adhesive compound. Alternatively, or in addition, the two pieces of the encasement 86 can snap together.

FIG. 8 illustrates bitewing mounting tab 34 in isolation. The bitewing mounting tab 34 includes an elongated slot 124 and a substantially square slot 128. The square slot 128 is formed in the mounting tab 34 as an aperture for the second arm 22a to be received within. The square slot 128 also includes an arm guide 132, which only allows the arm 22a to be received in the square slot 128 when inserted from one side of the bitewing mounting tab 34. The elongated slot 124 is provided to allow the arm guide 132 to move when the arm 22a is inserted into the square slot 128. While only the bitewing mounting tab 34 is described in detail above, it is to be understood that the description also applies for the A/P mounting tab 30, though the arrangement of the elongated slot 124 and the square slot 128 may be different for the A/P mounting tab 30 than it is for the bitewing mounting tab 34.

The ridge 14 of the encasement 86 also provides an alignment structure for the x-ray source 12. In the exemplary embodiment illustrated in FIGS. 2 and 3, the x-ray source 12 of an x-ray machine 16 includes a circular end 88 from which x-rays are emitted (FIGS. 2 and 3). However, the end 88 of the x-ray source need not be circular; other shapes—e.g., square or rectangular—can also be used. In any case, the end 88 of the x-ray source 12 is placed against the collimator portion 15 of the aiming ring 13. The perimeter of the x-ray source 12 engages the inner edge 116 of the ridge 14, which is annular in the illustrated embodiment but can have other shapes as well. By aligning the x-ray source 12 with the ridge 14, the emitted x-rays can be more easily and accurately directed towards the sensor 50.

In operation of the x-ray aiming device 10, an x-ray operator assembles the aiming device 10 with the correct sensor holder 26, 26a and arm 22, 22a according to the type of image or radiograph (e.g., periapical, bitewing, etc.) to be captured. Once the aiming device 10 is assembled, the x-ray operator helps positioned the sensor holder 26, 26a in a patient's mouth. Once the aiming device 10 is positioned correctly, the patient bites down on the biteblock 38, 38a. The biting force of the patient is sufficient to hold the aiming device 10 in the correct orientation. The x-ray operator then positions the x-ray machine 16 against the aiming ring 13, as described above. Once the x-ray machine 16 is correctly oriented, the x-ray operator can activate the x-ray machine 16 to begin the emission of x-ray radiation. Some of the emitted radiation passes through the aperture 18, through the patient's tissues, and impinges the sensor 50 to provide a radiograph or image. The remaining radiation emitted from the x-ray source 12 is substantially blocked by the x-ray shield 82 of the aiming ring 13.

Various features and advantages of the invention are set forth in the following claims.

What is claimed is:

1. An x-ray aiming device comprising:
an aiming ring including,
an x-ray shield, and
a plastic or elastomeric encasement covering the x-ray shield; and
an aperture in the x-ray shield allowing x-rays to pass through the x-ray aiming ring.

2. The x-ray aiming device of claim 1, further comprising:
at least one mounting tab formed as part of the encasement,
a sensor holder configured to hold an x-ray sensor; and
an arm configured to connect to both the mounting tab of the encasement and the sensor holder.

3. The x-ray aiming device of claim 2, wherein the mounting tab also includes an opening for receiving the arm for connection therewith.

4. The x-ray aiming device of claim 3, wherein the x-ray shield comprises tungsten.

5. The x-ray aiming device of claim 1, wherein the x-ray shield includes a plurality of notches.

6. The x-ray aiming device of claim 5, wherein the encasement includes a plurality of stabilizing holes that aligns with the plurality of notches.

7. The dental x-ray aiming ring of claim 1, further comprising a plurality of clamping holes formed in the encasement that are formed as a result of clamping the x-ray shield while molding the encasement.

8. The x-ray aiming device of claim 1, wherein the aperture is substantially shaped as an outline of two perpendicular elongated rectangles.

9. The x-ray aiming device of claim 1, wherein the encasement comprises at least one of polyetherimide and polyether ether ketone.

10. The x-ray aiming device of claim 1, wherein the encasement is formed with a ridge configured to align with an x-ray emitting device to aid in aiming emitted x-rays.

11. The x-ray aiming device of claim 10, wherein an outer circumference of the x-ray emitting device engages with an inner edge of the ridge of the encasement.

12. An x-ray aiming device comprising:
an aiming ring including,
an x-ray shield having an aperture, and
a plastic or elastomeric encasement covering the x-ray shield;
the aperture allowing x-rays to pass through the x-ray aiming ring;
at least one mounting tab formed as part of the encasement;
a sensor holder configured to hold an x-ray sensor; and
an arm configured to connect the sensor holder to the at least one mounting tab.

13. The x-ray aiming device of claim 12, wherein the sensor holder is configured to aid in capturing periapical radiographs.

14. The x-ray aiming device of claim 12, wherein the sensor holder is configured to aid in capturing bitewing radiographs.

15. The x-ray aiming device of claim 12, wherein the encasement includes a plurality of stabilizing holes that aligns with a plurality of notches in the x-ray shield.

16. The x-ray aiming device of claim 12, further comprising a plurality of clamping holes formed in the encasement that are formed as a result of clamping the x-ray shield while molding the encasement.

17. The x-ray aiming device of claim 12, wherein the encasement is formed with a ridge configured to align with an x-ray source.

18. The x-ray aiming device of claim 17, wherein an outer circumference of the x-ray device engages with an inner edge of the ridge of the encasement.

19. The x-ray aiming device of claim 12, further comprising a first indicia adjacent a first mounting tab and a second indicia adjacent a second mounting tab.
